(19) United States
(12) Patent Application Publication
(10) Pub. No.: US 2020/0224968 A1
(43) Pub. Date: Jul. 16, 2020

(54) Process and plant for separatory processing of a starting mixture

(71) Applicant: LINDE AKTIENGESELLSCHAFT, München (DE)

(72) Inventors: Tuat PHAM DUC, Penzberg (DE); Torben HÖFEL, München (DE)

(21) Appl. No.: 16/628,955

(22) PCT Filed: Jul. 6, 2018
(86) PCT No.: PCT/EP2018/068407

§ 371 Date: Jan. 6, 2020

(30) Foreign Application Priority Data
Jul. 6, 2017 (EP) 17180033.7

(51) Int. Cl. F25J 3/02; C01B 3/50; B01D 5/00; C07C 7/00; C07C 7/04; C07C 7/09

(52) U.S. Cl. CPC F25J 3/0219; F25J 2270/60; B01D 5/0003; B01D 5/0036; B01D 5/0054; B01D 5/0075; F25J 3/0233; F25J 3/0238; F25J 3/0252; C07C 7/005; C07C 7/04; C07C 7/09; F25J 2200/72; F25J 2205/04; F25J 2210/12; F25J 2230/32; F25J 2230/60; F25J 2270/02; C01B 3/506

(57) ABSTRACT

The present invention relates to a process (100) for separatory processing of a starting mixture containing predominantly hydrogen, methane and hydrocarbons having two or two or more carbon atoms, wherein at least a portion of the starting mixture is cooled to form one or more condensates using one or more heat exchangers (101, 103, 105, 107) and at least a portion of the condensate(s) is subjected to a rectification to form a gaseous methane-rich fraction. It is provided that the gaseous methane-rich fraction is used to form a first fluid stream which is at least partly compressed, in an unchanged composition with respect to the gaseous methane-rich fraction, to a liquefaction pressure level of 35 to 45 bar, and at least partly liquefied by cooling, and in that the first fluid stream, or a second fluid stream formed using the first fluid stream, is expanded to a delivery pressure and heated in the or at least one of the heat exchanger(s) (101, 103, 105, 107). A corresponding plant likewise forms part of the subject matter of the invention.

The invention relates to a process for separatory processing of a starting mixture containing predominantly hydrogen, methane and hydrocarbons having two or two and more carbon atoms and to a corresponding plant according to the preambles of the independent claims.

### PRIOR ART

Processes and plants for steam cracking of hydrocarbons are described for example in the article “Ethylene” in Ullmann's Encyclopedia of Industrial Chemistry, Online Edition, 15 Apr. 2007, DOI 10.1002/14356007.a10_045.pub2. Steam cracking is used, for example, to obtain short-chain olefins such as ethylene and propylene, diolefins such as butadiene or aromatics, but is not limited thereto.

Steam cracking affords a substance mixture initially described as crude gas. This is subjected to a plurality of processing steps, for example a crude gas scrubbing, a crude gas compression and a so-called primary fractionation. The crude gas processed in this way is subsequently supplied to a separation used to obtain components or component groups of the crude gas. There may also be provision for certain components of the crude gas to be reacted by hydrogenation or other processes upstream of or within such a separation.

A typical separation comprises a plurality of separating steps, each of which afford component groups. Known examples include “demethanizer first” (or “frontend demethanizer”), “deethanizer first” or (“frontend deethanizer”) or “depropanizer first” (or “frontend depropanizer”) processes. For details see the technical literature, for example the mentioned article “Ethylene” in Ullmann's Encyclopedia of Industrial Chemistry.

WO 2017/001514 A1 relates to a method for obtaining hydrogen from a gaseous feed mixture which is enriched with hydrogen, methane, and hydrocarbons with two carbon atoms. The fluid of the feed mixture is cooled from a first temperature level to a second temperature level at a first pressure level such that one or more condensates are separated from the fluid of the feed mixture while leaving a residual gas. The fluid of the residual gas is further cooled to a third temperature level and subjected to a countercurrent absorption at the first pressure level, thereby obtaining a hydrogen- and methane-enriched head gas and a bottom liquid. The fluid of the head gas is heated and subjected to a pressure swing adsorption at the first pressure level, thereby forming a hydrogen-enriched product flow which has a low methane content or which is methane-free, and the fluid of the condensate(s) and/or of the bottom liquid is expanded from the first pressure level to a second pressure level and fed to a low-pressure demethanizer at the second pressure level. According to the invention, the countercurrent absorption is carried out using fluid which is removed from the low-pressure demethanizer at the second pressure level, compressed to the first pressure level in the gaseous state, and cooled to the third temperature level. This document likewise relates to a corresponding system.

The present invention relates in particular to separations affording a substance mixture containing predominantly hydrogen, methane and hydrocarbons having two carbon atoms from which the hydrocarbons having two carbon atoms are initially obtained in a common fraction by removal of methane and lower boiling compounds such as hydrogen. Such a substance mixture containing predominantly hydrogen, methane and hydrocarbons having two carbon atoms or being rich in these components is hereinbelow also referred to as “starting mixture”. However, the present invention may in principle also be used for separatory processing of corresponding starting mixtures containing predominantly hydrogen, methane and hydrocarbons having two and more carbon atoms. In the former case the starting mixture is in the context of the present invention treated in a demethanizer of a “deethanizer first” process and in the latter case in a demethanizer of a “demethanizer first” process.

As also elucidated below, in certain cases prior art processes for separatory processing of such starting mixtures have proven unsatisfactory, in particular when hydrocarbons in a particular spectrum are subjected to the steam cracking process as inputs.

The present invention accordingly has for its object to improve and in particular carry out in a more energy efficient manner the separatory processing of such input mixtures.

### DISCLOSURE OF THE INVENTION

This object is achieved by a process for separatory processing of a starting mixture containing predominantly hydrogen, methane and hydrocarbons having two or two and more carbon atoms and to a corresponding plant having the features of the independent claims. Embodiments are in each case provided by the dependent claims and the description which follows.

Commonly used processes for separating product streams from processes for producing hydrocarbons such as the cracking gas mentioned at the outset comprise forming a series of fractions based on the different boiling points of the components present. These are referred to in the art by abbreviations indicating the carbon number of the hydrocarbons predominantly or exclusively present in each case. Thus a “C1 fraction” is a fraction containing predominantly or exclusively methane (and by convention in some cases also hydrogen, then also referred to as “C1minus fraction”). By contrast a “C2 fraction” contains predominantly or exclusively ethane, ethylene and/or acetylene. A “C3 fraction” contains predominantly propane, propylene, methylacetylene and/or propadiene. A “C3 fraction” contains predominantly or exclusively butane, butene, butadiene and/or butyne, wherein the respective isomers may be present in different proportions depending on the source of the C4 fraction. The same applies correspondingly for the “C5 fraction” and the higher fractions. Two or more such fractions may be subsumed. For example a “C2plus fraction” contains predominantly or exclusively hydrocarbons having two and more carbon atoms and a “C2minus fraction” contains predominantly or exclusively hydrocarbons having one and two carbon atoms and also optionally hydrogen and methane. Such fractions may also be employed as refrigerants, for example C2 or C3 fractions. Temperature levels achievable by means of corresponding C2 or C3 refrigerants are commonly also referred to as “C2 refrigeration” or “C3 refrigeration”. These refrigerants are conducted in refrigeration circuits where they are first compressed to a certain end-pressure level and starting therefrom subsequently expanded to different pressure levels for refrigeration generation at corresponding temperature levels.

In the parlance used here, liquid and gaseous streams or liquid or gaseous mixtures may be rich or poor in one or more components, wherein “rich” may represent a content of not less than 90%, 95%, 99%, 99.5%, 99.9%, 99.99% or 99.999% and “poor” may represent a content of not more than 10%, 5%, 1%, 0.1%, 0.01% or 0.001% on a molar, weight or volume basis. In the context of the terminology used here, liquid and gaseous streams or liquid or gaseous mixtures may further be enriched or depleted in one or more components, wherein these terms relate to a corresponding content in an original mixture from which the liquid or gaseous material stream or the liquid or gaseous mixture was obtained in each case. The liquid or gaseous material stream or the liquid or gaseous mixture is “enriched” when it contains not less than 1.1 times, 1.5 times, 2 times, 5 times, 10 times, 100 times or 1000 times the content and “depleted” when it contains not more than 0.9 times, 0.5 times, 0.1 times, 0.01 times or 0.001 times the content of a corresponding component based on the original mixture. A material stream or a corresponding mixture containing “predominantly” one or more components contains these one or more components at least to an extent of 80% or is rich in these in the previously elucidated sense.

For characterization of pressures and temperatures, the present application uses the terms “pressure level” and “temperature level”, which is intended to express the fact that corresponding pressures and temperatures in a corresponding plant need not be used in the form of exact pressure and temperature values in order to implement the concept of the invention. However, such pressures and temperatures typically vary within particular ranges of, for example, ±1%, 5%, 10%, 20% or even 50% around an average value. It is possible here for corresponding pressure levels and temperature levels to lie in disjoint ranges or in overlapping ranges. In particular, pressure levels encompass, for example, pressure drops that are unavoidable or to be expected, for example on account of cooling effects. The same holds for temperature levels. The pressure levels indicated here in bar are absolute pressures.

For the design and specific configuration of rectification columns and absorption columns, as may also be employed in the context of the present invention, reference is made to relevant textbooks (see for example Sattler, K.: Thermische Trennverfahren: Grundlagen, Auslegung, Apparate, 3rd Edition 2001, Weinheim, Wiley-VCH).

### Advantages of the Invention

The separatory treatment of the starting mixtures such as are processed in the context of the present invention conventionally employs exclusively or predominantly distillative processes which may be designed differently depending on the compounds converted in the steam cracking and the corresponding composition of the starting mixtures. Common to the processes is that temperatures of −100° C. and less are used. Different processes can be used to generate such low temperatures. The use of cascaded refrigeration circuits is known for example. This may involve the use of C3 refrigerant(s) (propane and/or propylene down to about −40° C.) followed by a C2 refrigerant(s) (ethylene down to about −100° C.) and subsequently methane (C1) or mixed refrigerant(s) (down to about −160° C.). Such starting mixtures may also be cooled using cold fractions or product streams formed from the starting mixtures themselves. The latter may be suitably expanded in known processes to obtain refrigeration and subsequently recompressed in a crude gas compressor, thus allowing “open” refrigerant circuits to be formed. A combination of these measures is also possible.

In a number of known processes a methane- and hydrogen-containing fraction removed from a corresponding input mixture is used as refrigerant and at a low pressure level discharged at the plant limits as a so-called tail gas. However, this is not always desired, in particular when using steam cracking processes in which liquid inputs such as naphtha or mixed inputs, which, however, comprise such liquid inputs, are supplied. What is often desired here is to generate a separate hydrogen-rich fraction at a high pressure level and a methane-rich fraction at a low pressure level.

The methane-rich fraction can be sent for energy recovery as tail gas/fuel gas, which typically requires only a low pressure level, and the separate hydrogen-rich fraction may be sent for material use, for example for hydrogenations in the plant and/or as a value-adding plant product, which typically requires a high pressure level. When using corresponding inputs, this objective may typically be achieved by employing conventional C3 and C2 refrigeration circuits (as a cascade) and with the aid of a cold, liquid methane product fraction formed, as coolant. Further measures such as the use of C1 or mixed refrigerants, expanders or recycle streams to the crude gas compression are not required.

However, separately obtaining a hydrogen-rich fraction at a high pressure level becomes increasingly difficult the higher the hydrogen proportion relative to the methane proportion in a corresponding starting mixture sent for separatory processing. This ratio of hydrogen to methane in the starting mixture in turn depends on the inputs going into the steam cracking. In the case of predominantly liquid inputs, the ratio of hydrogen to methane in the starting mixture entering the separatory processing is typically below 1 mol/mol. Elevated hydrogen proportions are obtained in “mixed feed” steam cracking processes, i.e. in steam cracking processes in which not only gaseous inputs such as ethane but also the previously elucidated liquid inputs are subjected to steam cracking. Depending on the mixture of the input materials in such steam cracking processes, the ratio of hydrogen to methane in the output mixture entering the separatory processing is for example between 1 mol/mol and 2 mol/mol, in particular between 1.2 mol/mol and 1.8 mol/mol.

In such cases the amount of the liquid, cold methane product fraction formed is available as coolant in an insufficient amount and it is therefore necessary to resort to other options. The use of closed C1 or mixed refrigerant circuits, while conceivable in principle, is complex and costly and recycle streams to the crude gas compression are energy inefficient. If an expander were employed, for example, this would be at the expense of the obtainable hydrogen product amount.

The present invention makes use of the fact that in conventional processes of the elucidated type a gaseous methane product fraction is typically also formed in addition to the liquid methane product fraction. Reference is made in this regard to the accompanying FIG. 1. As also elucidated again hereinbelow, the process illustrated therein initially forms a liquid, methane-rich fraction in the form of the bottoms liquid from the separation vessel 108 which is subsequently provided in the form of the material stream M and a gaseous methane-rich fraction in the form of the tops gas from the separation vessel 113 which is subsequently provided in the form of the material stream Q.

In the context of the present invention the gaseous methane fraction mentioned is at least in part, in an unchanged composition, brought to a pressure high enough to ensure that substantial portions of this methane fraction can be condensed using C2 refrigeration. Liquid methane formed in this way is then available as additional refrigerant and the objective of generating a separate hydrogen-rich fraction at a high pressure level can be achieved. This requires only a low pressure ratio of less than 1.4 or 1.6. A relatively small, low output apparatus (magnitude of 1% to 5% of the output of the crude gas condenser) may therefore be employed. This is possible because the gaseous methane fraction leaves the employed rectification column slightly below the level of C2 refrigeration (about −97° C.). This gaseous methane fraction need therefore be further compressed only to a relatively small extent (to about 35 to 45 bar) in order to be able in any case to largely condense said fraction at the cited temperature level using C2 refrigeration. The condensation is carried out in order that the obtained condensate or a stream generated therefrom may subsequently be utilized in a heat exchanger at a temperature level below C2 refrigeration. This requires that the condensate is expanded before, for example to 7 bar. After this expansion, the corresponding obtained refrigeration may be utilized at the lower temperature level. The process according to the invention accordingly makes it possible to obtain a large amount of a hydrogen-rich fraction at a high pressure level with little additional complexity.

As a precaution it is noted here that in contrast to the present invention the prior art elucidated for example in respect of FIG. 2 of WO 2015/104153 A1 relates to a case where a liquid methane fraction is conveyed by means of a pump. A gaseous methane stream is not compressed here, but rather merely utilized as a gaseous refrigerant at a higher temperature level than in the context of the present invention. The conveyed methane fraction is subsequently used as reflux on a C2 absorber.

The heat exchangers 1 to 3 shown in FIG. 2 of WO 2015/104153 A1 are balanced by C2 refrigeration, the heat exchanger 4 is cooled only with product streams. When not enough liquid methane is available (i.e. there is a high ratio of hydrogen to methane) then according to the invention and in contrast to WO 2015/104153 A1 the stream I shown there is brought to a pressure high enough to ensure that said stream can already be predominantly condensed against C2 refrigeration in the heat exchanger in order that said stream is subsequently usable as refrigerant in the heat exchanger 4. In the context of the present invention a conventional refrigeration plant (compression, condensation, expansion, evaporation) is therefore concerned. However, the invention employs an “open refrigeration generation” rather than an “open refrigeration circuit”, since each methane molecule from the stream I is passed through the compressor and subsequently expanded to the low tail gas pressure only precisely once.

The present invention proceeds from a process for separatory processing of a starting mixture containing predominantly hydrogen, methane and hydrocarbons having two or two or more carbon atoms, wherein at least a portion of the starting mixture is cooled to form one or more condensates using one or more heat exchangers and at least a portion of the condensate(s) is subjected to a rectification to form a gaseous methane-rich fraction. Typically effected in the context of the present invention as also elucidated hereinbelow is a stage-wise cooling with respective separation of a plurality of condensates and introduction thereof into a rectification column.

In the context of the process according to the invention the gaseous methane-rich fraction is formed in particular at a temperature level of −95° C. to −100° C., preferably −96° C. to −98° C. This temperature level is close to that of C2 refrigeration and a relatively small subsequent compression is therefore sufficient to subsequently be able to at least partly condense the gaseous fraction as mentioned.

It is explicitly emphasized that it is also possible in the context of the present invention to employ, in particular instead of a plurality of separate heat exchangers, constructionally integrated heat exchanger stages in a common heat exchanger, which may be configured for example in the form of a plurality of heat exchanger sections/heat exchanger blocks in fluid communication with one another. Stage-wise cooling and formation of a plurality of condensates is also possible in a heat exchanger of this type. Although not elucidated in detail hereinbelow, the cooling may in principle also be performed using only one cooling stage with formation of only one condensate.

In the context of the invention, the elucidated cooling is in principle effected under pressure and temperature conditions (see also below in this regard) by means of which it may be ensured that after the cooling and the formation of the condensate(s) a residue remaining in gaseous form contains only a small amount, if any, of hydrocarbons having two and optionally more hydrocarbons. This ensures that no valuable product or compounds recyclable into the steam cracking are still present in the residue remaining in gaseous form and thus lost or otherwise recoverable only in very complex fashion. Such a cooling and formation of the condensate(s) is in particular effected such that a residue remaining in gaseous form after the separation of the condensate(s) contains not more than 1 mol %, preferably not more than 0.1 mol %, particularly preferably not more than 0.01 mol %, of hydrocarbons having two or optionally more carbon atoms.

The formation of the condensate(s) is thus carried out with a view to largely depleting hydrocarbons having two or optionally more carbon atoms. It is a corollary of this that certain amounts of methane and hydrogen are together with the hydrocarbons having two or optionally more carbon atoms likewise separated from such a starting mixture into the condensate(s). The chief purpose of the abovementioned rectification is to remove the methane and the hydrogen from hydrocarbons having two or optionally more carbon atoms. As mentioned, this can be problematic in particular when using mixed inputs in a steam cracking process, since for increasing ratios of hydrogen to methane in the condensates entering the rectification an ever higher column pressure and/or an ever lower temperature in the tops condenser of the distillation are required.

The process according to the invention may therefore be employed in particular when the starting mixture entering the separatory processing has a ratio of hydrogen to methane of for example between 1 mol/mol and 2 mol/mol, in particular between 1.2 mol/mol and 1.8 mol/mol. Such a starting mixture may be obtained in particular from a cracking gas/crude gas from a steam cracking process supplied with the previously elucidated mixed inputs. For conventional ratios of hydrogen to methane below 1 mol/mol, conventional separation processes typically known from plants for steam cracking of liquid inputs may be used. For ratios of hydrogen to methane above 2 mol/mol, conventional separation processes typically known from plants for steam cracking of ethane may be used, for example utilizing the refrigeration in the hydrogen-rich fraction which can then no longer be discharged at the plant limits as a high-pressure fraction. By contrast, the present invention relates in particular to the specific case of using mixed inputs in steam cracking.

According to the invention it is provided that the gaseous methane-rich fraction is at least partly used to form a first fluid stream which is compressed, in an unchanged composition with respect to the gaseous methane-rich fraction, to a liquefaction pressure level of 35 to 45 bar, preferably of 35 to 40 bar, particularly preferably 35 to 38 bar, at least partly liquefied by cooling, and is, in a still unchanged composition with respect to the gaseous methane-rich fraction, expanded to a lower pressure level, here also referred to as the “delivery pressure level”. If reference is made herein to the first fluid stream, i.e. the part of the methane-rich fraction which is compressed, at least partially liquefied and subsequently expanded, being subjected to these steps “in an unchanged composition with respect to the gaseous methane-rich fraction”, this shall express that the relative proportions of components, irrespectively whether they are present in liquid, gaseous or mixed-phase form, do not change in this fluid stream as a whole. In particular, the first fluid stream is not be mixed with other fluid streams, changing its composition, and is not subjected to any separation or step by being separated into a gas phase and a liquid phase with different compositions, only one of these phases being further used. In particular, this first fluid stream is not fed into an absorption column and is subjected to a depletion or enrichment of components to form further fluid streams of different composition therein. In particular the process steps of compression, liquefaction and expansion are direct in succession, in particular without intermediate further steps (apart from, for example, heating and cooling or transfer through pipes). However, this does not exclude the possibility that the first fluid stream may be in the form of a two-phase stream with a liquid phase and a gaseous phase if it is not completely liquefied. However, the overall composition of the first fluid stream, which is the sum of the compositions in the liquid and gaseous phases, also changes in this case not, even if the individual compositions of the two phases should be different. The first fluid stream, or a second fluid stream formed using the first fluid stream, is, subsequently to the first fluid stream having been expanded to the delivery pressure level, at which he still has the same composition as the gaseous methane-rich fraction, heated in the or at least one of the heat exchanger(s).

The first fluid stream, or a second fluid stream formed using the first fluid stream, is, subsequently to the first fluid stream having been expanded to the delivery pressure level, at which he still has the same composition as the gaseous methane-rich fraction, heated in the or at least one of the heat exchanger(s). This makes it possible, as elucidated hereinabove, to provide a hydrogen-rich, gaseous product fraction at a sufficiently high pressure level irrespective of the lower methane amounts. This is achieved in particular when, during cooling of the starting mixture or of the portion thereof in the heat exchanger(s), heat is transferred to the compressed and at least partially liquefied methane-rich fraction or the portion thereof in the heat exchanger(s).

The cooling of the starting mixture or of the portion thereof in the heat exchanger(s) is preferably performed at a cooling pressure level below the liquefaction pressure level. The hydrogen-rich, gaseous product fraction which preferably need not be subjected to any expansion in the context of the process according to the invention may also be provided at said pressure level. By contrast, in the context of the present invention the rectification is preferably performed at a rectification pressure level slightly below the cooling pressure level in order that the condensates may be passed to the rectification without a pump. The cooling pressure level is in particular 25 to 40 bar, preferably 30 to 38 bar, particularly preferably 32 to 38 bar. In the context of the invention, the rectification pressure level is in particular 0.2 to 4 bar, preferably 1 to 3 bar, particularly preferably 2 to 3 bar below the cooling pressure level.

As already mentioned more than once it is possible in the context of the present invention to provide a hydrogen-rich product fraction at a satisfactory pressure level in particular irrespective of the low contents of methane in a corresponding starting mixture. A process according to a particularly preferred embodiment of the process according to the invention therefore comprises likewise heating in the or at least one of the heat exchanger(s) a hydrogen-rich fraction remaining in gaseous form in the cooling of the starting mixture or of the portion thereof in the heat exchanger(s). The hydrogen-rich fraction thus remains, in particular downstream of a cooling step, at a temperature level of −120° C. to −170° C., preferably −140° C. to −165° C., for example about −160° C., at which at least a predominant proportion of methane condenses out. Sufficiently low temperatures are achievable in particular by using a methane-rich refrigerant, in the context of the present invention inter alia using the compressed and at least partially liquefied methane-rich fraction. The fraction remaining in gaseous form thus contains in particular 80 to 100 mol %, preferably 85 to 95 mol %, for example about 90 mol %, of hydrogen.

In order to provide a corresponding hydrogen-rich product fraction, the present invention in particular provides for heating the hydrogen-rich fraction in an unexpanded state. In the context of the present invention, the heating of the hydrogen-rich fraction or the portion thereof is therefore preferably undertaken at the cooling pressure level.

As mentioned, in the context of the present invention the compressed and at least partly liquefied methane-rich fraction or the portion thereof (i.e. the “first fluid stream” or the “second fluid stream” mentioned hereinbefore) is expanded to a delivery pressure level below the cooling pressure level before the heating in the heat exchanger(s). The delivery pressure level may be for example 2 to 10 bar, in particular 5 to 8 bar, in particular about 7 bar. In this way refrigeration may be generated and in the context of the present invention utilized for the starting mixture/the employed proportion thereof.

Further methane-rich fractions may likewise be employed in the context of the present invention. Thus according to a particularly advantageous embodiment of the process according to the invention the rectification may afford a liquid, methane-rich fraction which is at least partly heated in the heat exchanger(s) together with the compressed and at least partly liquefied methane-rich fraction or the portion thereof. A fraction of this kind may thus be utilized appropriately even if present in a relatively small proportion.

In detail, a process such as is also elucidated with reference to the accompanying FIG. 2 may be particularly advantageous. This comprises carrying out a cooling using a first heat exchanger, a second heat exchanger, a third heat exchanger and a fourth heat exchanger which are connected in series and through which the starting mixture or the employed proportion thereof are passed to effect continuous depletion of the hydrocarbons having two or optionally more carbon atoms (and also methane and small amounts of hydrogen). In a process according to a particularly preferred embodiment of the present invention the starting mixture or the portion thereof is thus passed consecutively through the first, the second, the third and the fourth heat exchanger, a respective condensate being separated downstream of each heat exchanger. It is advantageous when the formation of the condensates is effected by purely condensative means, i.e. without absorption columns as in the prior art.

In the context of the present invention it is in particular possible to operate the first heat exchanger using an ethylene-rich refrigerant (for example so-called high-pressure ethylene) at −50° C. to −60° C., preferably about −57° C., the second heat exchanger using an ethylene-rich refrigerant (for example so-called mid-pressure ethylene) at −75° C. to −85° C., preferably about −80° C., and the third heat exchanger using an ethylene-rich refrigerant (for example so-called low-pressure ethylene) at −95° C. to −105° C., preferably about −100° C. Corresponding temperature levels are employed in the context of the elucidated cooling pressure level.

As mentioned, it is possible in the context of the present invention to employ fractions of the starting mixture in a final cooling step. The present invention in particular provides for using the fourth heat exchanger to heat fractions of a fraction that remains in gaseous form after cooling in the third heat exchanger and has previously been cooled in the fourth heat exchanger. This makes it possible in the context of the present invention to achieve in particular a temperature level of −120° C. to −180° C., in particular about −160° C.

In the context of the previously elucidated embodiment of the process according to the invention it is in particular provided that the gaseous methane-rich fraction or the employed portion thereof (the “first fluid stream” mentioned hereinbefore) is consecutively heated in the third heat exchanger, passed through a further heat exchanger, compressed to the liquefaction pressure level, passed through the further heat exchanger and cooled in the third and fourth heat exchanger. The now partly or fully liquefied, previously gaseous, methane-rich fraction is subsequently heated in the fourth, the third, the second and the first heat exchanger.

The present invention also relates to a plant for separatory processing of a starting mixture containing predominantly hydrogen, methane and hydrocarbons having two or two or more carbon atoms, comprising means for cooling at least a portion of the starting mixture to form one or more condensates using one or more heat exchangers and for subjecting at least a portion of the condensate(s) to a rectification to form a gaseous methane-rich fraction. Provided according to the invention are means which are adapted to use the methane-rich fraction to form a first fluid stream and means which are adapted to at least partly compress the first fluid stream, in an unchanged composition with respect to the gaseous methane-rich fraction, to a liquefaction pressure level of 35 to 45 bar, in particular the abovementioned values, to at least partly liquefie it by cooling, and to expand it to a to a delivery pressure level. Further means are provided which are adapted to heat the first fluid stream or a second fluid stream which is formed using the first fluid stream in the or at least one of the heat exchanger(s).

For features and advantages of a corresponding plant advantageously adapted for performing a process, as previously elucidated in the embodiments, express reference is made to the above explanations.

One embodiment of the invention is hereinbelow more particularly elucidated with regard to the prior art with reference to the accompanying drawings.

### BRIEF DESCRIPTION OF THE DRAWINGS

FIG. 1 shows a non-inventive process in the form of a schematic process flow diagram.

FIG. 2 shows a process according to one embodiment of the invention in the form of a schematic process flow diagram.

### DETAILED DESCRIPTION OF THE DRAWINGS

In the figures that follow, mutually corresponding elements bear corresponding reference numerals and for the sake of simplicity are not repeatedly elucidated.

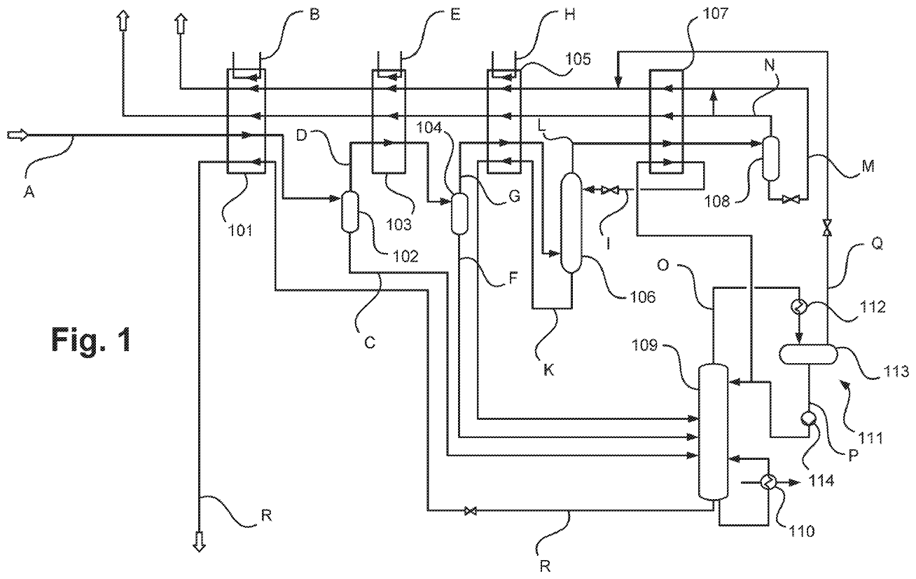

FIG. 1 illustrates a non-inventive process for separatory processing of a starting mixture containing predominantly hydrogen, methane and hydrocarbons having two carbon atoms in the form of a schematic process flow diagram to illustrate the background to the present invention. The process is employed in particular for separatory processing of a starting mixture formed from a crude gas from a steam cracking process where predominantly or exclusively liquid inputs, for example naphtha, are processed.

It is expressly emphasized that FIG. 1 serves merely to illustrate the principles of the present invention and it is quite possible to deviate from the specific configuration of the depicted apparatuses shown here without providing a fundamentally different separation process.

The starting mixture or a portion thereof in the form of the material stream A is initially passed through a first heat exchanger 101 and cooled inter alia using a material stream B which may be for example ethylene at a temperature level of about −57° C.

The material stream A is transferred into a first separation vessel 102 from which a liquid material stream C and a gaseous material stream D are withdrawn. The gaseous material stream D is passed through a second heat exchanger 103 and here cooled further inter alia with a material stream E which may be ethylene at a temperature level of about −80° C.

The material stream D is transferred into a further separation vessel 104 from which a liquid material stream F and a gaseous material stream G are withdrawn. The material stream G is passed through a third heat exchanger 105 and there cooled inter alia with a material stream H which may be ethylene at a temperature level of about −100° C.

The material stream G is subsequently transferred into an absorption column 106 which is operated with a methane-rich liquid material stream I as reflux. A liquid material stream K is withdrawn from the bottom of the absorption column 106 and, in the example shown, heated in the heat exchanger 105.

A gaseous material stream L from the top of the absorption column 106 is cooled further in a heat exchanger 107 and subsequently transferred into a further separation vessel 108, the so-called hydrogen separator. A liquid methane-rich material stream M and a gaseous hydrogen-rich material stream N are withdrawn from the separation vessel 108. The material stream M is expanded to a lower pressure level; the material stream N remains at the higher pressure level at which it is withdrawn from the separation vessel 108. Both material streams are heated in the heat exchangers 107, 105, 103 and 101 and provided as the methane-rich product fraction and the hydrogen-rich product fraction respectively.

The abovementioned liquid material streams C, F and K are transferred into a rectification column 109, the introduction thereof being effected at different heights depending on composition and temperature. The rectification column 109 is operated with a bottoms evaporator 110 using a typical C3 refrigerant. A gaseous material stream O is withdrawn from the top of the rectification column 109 and supplied to a tops condenser having the overall designation 111. The tops condenser 111 comprises a heat exchanger 112 which may be operated with ethylene at a temperature level of about −100° C. as the refrigerant. Obtained in a separation vessel 113 arranged downstream of the heat exchanger 112 a liquid material stream P, one portion of which is applied as reflux to the rectification column 109 and one portion of which is applied as reflux to the absorption column 106 in the form of material stream I. A non-liquefied proportion of the material stream O is withdrawn from the separation vessel 113 in the form of the material stream Q and as a methane-rich material stream combined with the material stream M.

The process illustrated in FIG. 1 thus initially forms a liquid, methane-rich fraction in the form of the bottoms liquid from the separation vessel 108, which is subsequently provided in the form of the material stream M, and a gaseous methane-rich fraction in the form of the tops gas from the separation vessel 113, which is subsequently provided in the form of the material stream Q.

The elucidated operation of the rectification column 109 makes it possible to withdraw from the bottom thereof a liquid material stream R which is rich in hydrocarbons having two carbon atoms. In the example shown said stream is heated in the heat exchanger 101 and subsequently for example subjected to a separation to obtain ethane and ethylene in a so-called C2 splitter.

As previously elucidated, separately obtaining a hydrogen-rich fraction at a high pressure level becomes increasingly difficult the higher the hydrogen proportion relative to the methane proportion in a corresponding starting mixture, since the heat exchanger 107 is cooled exclusively with product streams and the heat balance around the heat exchanger 107 becomes increasingly unfavourable the higher the hydrogen proportion relative to the methane proportion in a corresponding input mixture.

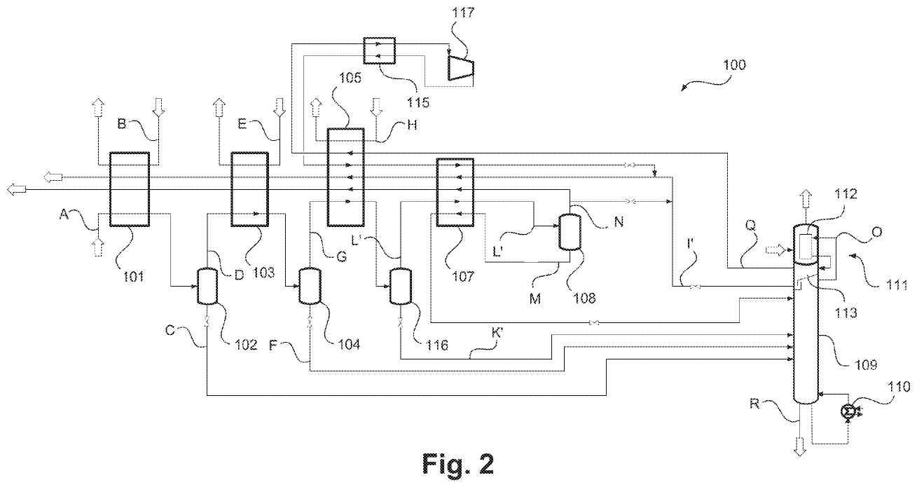

Illustrated in FIG. 2 in the form of a schematic process flow diagram and designated 100 is a process for separatory processing of such a starting mixture according to one embodiment of the invention. The process 100 is employed in particular for separatory processing of a starting mixture formed from a crude gas from a steam cracking process where a mixed input, as elucidated hereinabove, is processed.

Here too, the starting mixture or a portion thereof in the form of a material stream A is initially passed through a first heat exchanger 101 and cooled inter alia using a material stream B which may be for example ethylene at a temperature level of about −57° C.

Here too, the material stream A is transferred into a first separation vessel 102 from which a liquid material stream C and a gaseous material stream D are withdrawn. Here too, the gaseous material stream is passed through a second heat exchanger 103 and here cooled further inter alia with a material stream E which may be ethylene at a temperature level of about −80° C.

Here too, the material stream D is transferred into a further separation vessel 104 from which a liquid material stream F and a gaseous material stream G are withdrawn. Here too, the material stream G is passed through a third heat exchanger 105 and there cooled inter alia with a material stream H which may be ethylene at a temperature level of about −100° C.

However, in contrast to the process illustrated in FIG. 1, the material stream G is now transferred not into an absorption column 106 but rather into a further separation vessel 116, from which a liquid material stream K′ and a gaseous material stream L′ are withdrawn. The material stream L′ is cooled further in a heat exchanger 107 and subsequently transferred into a further separation vessel 108 which here too constitutes a hydrogen separator.

Here too, a liquid methane-rich material stream M and a gaseous hydrogen-rich material stream N are withdrawn from the separation vessel 108. The material stream M, the amount of which is limited by a valve not separately designated here, is heated in the heat exchanger 107. In the example shown only the material stream N is consecutively heated in the heat exchangers 107, 105, 103 and 101 at the cooling pressure level and provided as a hydrogen-rich product fraction.

The abovementioned liquid material streams C, F and K′ and also the material stream M are transferred into a rectification column 109 under limitation of valves not separately designated here, the introduction of said streams being effected at different heights depending on composition and temperature. Here too, the rectification column 109 is operated with a bottoms evaporator 110 using a typical C3 refrigerant. As before, a gaseous material stream O is withdrawn from the top of the rectification column 109 and supplied to a tops condenser having the overall designation 111. However, the tops condenser 111 is here integrated into the rectification column 109. Said tops condenser comprises a heat exchanger 112 which may be operated with ethylene at a temperature level of about −100° C. as the refrigerant. Accumulating in a separation vessel 113 connected downstream of the heat exchanger 112 but here likewise integrated into the rectification column 109 is a liquid fraction which is here applied to the rectification column 109 without a pump but rather via an overflow. Since there is no absorption column present, no reflux is required therefor. Thus after expansion a liquid material stream I′ is supplied to a heating in the heat exchangers 107, 105, 103 and 101 and discharged from the process 100.

Here too, a non-liquefied proportion of the material stream O is withdrawn from the separation vessel 113 in the form of the material stream Q but now is initially heated into the heat exchanger 105, subsequently passed through a further heat exchanger 115 and compressed in a booster 117. Subsequently, the material stream Q is again passed through the heat exchangers 117, 105 and 107, expanded, combined with the material stream I′ and finally heated in the heat exchangers 107, 105, 103 and 101 and discharged from the process 100.

Here too, the elucidated operation of the rectification column 109 makes it possible to withdraw from the bottom thereof a liquid material stream R which is rich in hydrocarbons having two carbon atoms. In the process 100 too, said stream is for example subjected to a separation to obtain ethane and ethylene in a so-called C2 splitter.

1. Process (100) for separatory processing of a starting mixture containing predominantly hydrogen, methane and hydrocarbons having two or two or more carbon atoms, wherein at least a portion of the starting mixture is cooled to form one or more condensates using one or more heat exchangers (101, 103, 105, 107) and at least a portion of the condensate(s) is subjected to a rectification to form a gaseous methane-rich fraction, characterized in that the methane-rich fraction is used to form a first fluid stream which is at least partly compressed, in an unchanged composition with respect to the gaseous methane-rich fraction, to a liquefaction pressure level of 35 to 45 bar, is at least partly liquefied by cooling, and is expanded to a delivery pressure level, and in that the first fluid stream, or a second fluid stream formed using the first fluid stream, is heated in the or at least one of the heat exchanger(s) (101, 103, 105, 107).

2. Process (100) according to claim 1, wherein the gaseous methane-rich fraction is formed at a temperature level of −95° C. to −100° C.

3. Process (100) according to claim 1, wherein the cooling of the starting mixture or of the portion thereof in the heat exchanger(s) (101, 103, 105, 107) comprises the transferring of heat to the compressed and at least partially liquefied methane-rich fraction or the portion thereof.

4. Process (100) according to claim 1, wherein the cooling of the starting mixture or of the portion thereof in the heat exchanger(s) (101, 103, 105, 107) is performed at a cooling pressure level below the liquefaction pressure level of the methane-rich fraction.

5. Process (100) according to claim 4, wherein the cooling is performed at a cooling pressure level of 25 to 40 bar and wherein the rectification is performed at a rectification pressure level 0.2 to 4 bar below the cooling pressure level.

6. Process (100) according to claim 5, wherein a hydrogen-rich fraction remaining in gaseous form in the cooling of the starting mixture or of the portion thereof in the heat exchanger(s) (101, 103, 105, 107) is likewise heated in the or at least one of the heat exchanger(s).

7. Process (100) according to claim 6, wherein the hydrogen-rich fraction or the portion thereof is heated at the cooling pressure level.

8. Process according to claim 1, wherein the rectification affords a liquid, methane-rich fraction which is at least partly heated in the heat exchanger(s) (101, 103, 105, 107) together with the compressed and at least partly liquefied methane-rich fraction or the portion thereof.

9. Process according to claim 1, wherein the cooling is performed using a first heat exchanger (101), a second heat exchanger (103), a third heat exchanger (105) and a fourth heat exchanger (107).

10. Process according to claim 9, which comprises operating the first heat exchanger (101) using an ethylene-rich refrigerant at −50° C. to −60° C., the second heat exchanger (103) using an ethylene-rich refrigerant at −75° C. to −85° C. and the third heat exchanger (105) using an ethylene-rich refrigerant at −95° C. to −105° C.

11. Process according to claim 9, wherein the starting mixture or the portion thereof is passed consecutively through the first, the second, the third and the fourth heat exchanger (101, 103, 105, 107), a respective condensate being separated downstream of each heat exchanger.

12. Process according to claim 11, wherein fractions of a fraction that remains in gaseous form after cooling in the third heat exchanger (105) and has previously been cooled in the fourth heat exchanger (107) are heated using the fourth heat exchanger (107).

13. Process according to any of claims 9 to 12, wherein the methane-rich fraction or the portion thereof is consecutively heated in the third heat exchanger (105), passed through a further heat exchanger (115), compressed to the liquefaction pressure level, passed through the further heat exchanger (115) and cooled in the third and fourth heat exchanger (105, 107).

14. Plant for separatory processing of a starting mixture containing predominantly hydrogen, methane and hydrocarbons having two or two or more carbon atoms, comprising means for cooling at least a portion of the starting mixture to form one or more condensates using one or more heat exchangers (101, 103, 105, 107) and for subjecting at least a portion of the condensate(s) to a rectification to form a gaseous methane-rich fraction characterized by means which are adapted to use the gaseous methane-rich fraction to form a first fluid stream, by means by which are adapted to compress the first fluid stream at least partly to a liquefaction pressure level of 35 to 40 bar, to at least partly liquefy it by cooling, and to expand it to a delivery pressure level, and by means which are adapted to heat the first fluid stream, or a second fluid stream formed using the first fluid stream, in the or at least one of the heat exchanger(s) (101, 103, 105, 107).

15. Process (100) according to claim 2, wherein the cooling of the starting mixture or of the portion thereof in the heat exchanger(s) (101, 103, 105, 107) comprises the transferring of heat to the compressed and at least partially liquefied methane-rich fraction or the portion thereof.

16. Process (100) according to claim 2, wherein the cooling of the starting mixture or of the portion thereof in the heat exchanger(s) (101, 103, 105, 107) is performed at a cooling pressure level below the liquefaction pressure level of the methane-rich fraction.

17. Process (100) according to claim 3, wherein the cooling of the starting mixture or of the portion thereof in the heat exchanger(s) (101, 103, 105, 107) is performed at a cooling pressure level below the liquefaction pressure level of the methane-rich fraction.

18. Process according to claim 2, wherein the rectification affords a liquid, methane-rich fraction which is at least partly heated in the heat exchanger(s) (101, 103, 105, 107) together with the compressed and at least partly liquefied methane-rich fraction or the portion thereof.

19. Process according to claim 3, wherein the rectification affords a liquid, methane-rich fraction which is at least partly heated in the heat exchanger(s) (101, 103, 105, 107) together with the compressed and at least partly liquefied methane-rich fraction or the portion thereof.

20. Process according to claim 4, wherein the rectification affords a liquid, methane-rich fraction which is at least partly heated in the heat exchanger(s) (101, 103, 105, 107) together with the compressed and at least partly liquefied methane-rich fraction or the portion thereof.
